(19) United States
(12) Patent Application Publication
(10) Pub. No.: US 2013/0035465 A1
(43) Pub. Date: Feb. 7, 2013

(54) RADICAL POLYMERIZABLE RESIN, RADICAL POLYMERIZABLE RESIN COMPOSITION, AND CURED PRODUCT THEREOF

(71) Applicants: Naoko Araki, Himeji-shi (JP); Yoshinori Funaki, Himeji-shi (JP); Kiyoharu Tsutsumi, Himeji-shi (JP); Tomoaki Mahiko, Himeji-shi (JP)

(72) Inventors: Naoko Araki, Himeji-shi (JP); Yoshinori Funaki, Himeji-shi (JP); Kiyoharu Tsutsumi, Himeji-shi (JP); Tomoaki Mahiko, Himeji-shi (JP)

(73) Assignee: DAICEL CORPORATION, Osaka (JP)

(21) Appl. No.: 13/641,354

(22) PCT Filed: Apr. 8, 2011
(86) PCT No.: PCT/JP2011/058876

§ 371 Date: Oct. 15, 2012

(30) Foreign Application Priority Data
Apr. 16, 2010 (JP) 2010-095034

(51) Int. Cl. C07D 305/06; C08G 63/66; C08F 124/00

(52) U.S. Cl. 526/266; 528/361; 549/510

(57) ABSTRACT

Provided is a radically polymerizable resin which has a low viscosity, has excellent workability, and is rapidly cured upon application of heat and/or light to form a cured product having excellent flexibility and thermal stability. The radically polymerizable resin composition is obtained by cationic polymerization of an oxetane-ring-containing (meth)acrylic ester compound represented by following Formula (1) alone or in combination with another cationically polymerizable compound. In Formula (1), R¹ represents hydrogen atom or methyl group; R² represents hydrogen atom or an alkyl group; and “A” represents a linear or branched chain alkylene group having 4 to 20 carbon atoms.

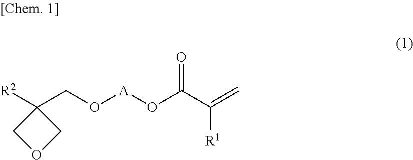

### TECHNICAL FIELD

The present invention relates to radically polymerizable resins (free-radically polymerizable resins or radical polymerizable resins), radically polymerizable resin compositions, and cured products obtained therefrom. They are useful in the fields of waveguides (e.g., optical waveguides and hybrid substrates), optical fibers, stress-relaxation adhesives, sealants, underfill materials, ink-jet inks, color filters, nanoimprinting materials, and flexible substrates (flexible boards); and are particularly useful in the fields of flexible optical waveguides, flexible adhesives, and underfill materials.

### BACKGROUND ART

Boards (substrates) used as servers or routers have increasing channel capacities with widespread proliferation of video distribution via internet. To meet this, replacement of part of high-speed signal lines from an electric wiring (electric interconnection) to an optical wiring (optical interconnection) has been more and more studied. Polymer optical waveguides are available at lower cost than that of quartz optical waveguides and are therefore expected as optical interconnections for opto-electric hybrid substrates. Thermal stability upon solder reflow is one of properties required of such optical interconnections. Specifically, solder reflow in a solder reflow process to form an opto-electric hybrid (wiring) board applies heat on a waveguide formed as an optical interconnection on a board, and the heat may cause thermal degradation (e.g., cracking and increase in optical loss) of the waveguide. To avoid this, the waveguide should have satisfactory thermal stability upon solder reflow. Recent solder reflow processes have been performed at higher temperatures, because lead-free solder, requiring heating at a high temperature of about 260° C. to be melt, has been employed. To meet this, the optical interconnections should have further satisfactory thermal stability upon solder reflow.

A technique of vertically stacking two or more semiconductor devices or wafers has been studied. According to this technique, two or more plies of a semiconductor device or wafer are stacked and bonded through an adhesive to give a stack, and a through hole penetrating the stack is provided to form a through-silicon via. Thus, electrodes vertically connected to each other are provided with a higher degree of vertical integration. However, when a material constituting the adhesive has a coefficient of thermal expansion different from that of a material constituting the adherend semiconductor device, wafer, or through-silicon via, the two materials constituting the adherend and the adhesive thermally expand (or contract) in different degrees upon heating or cooling to cause a stress. The stress may cause peeling (separation) at the adhesive interface between the adhesive and the adherend. In addition, the semiconductor device, wafer, and through-silicon via are thin-walled, fragile, and thereby liable to break when receiving external force applied as a result of heating or cooling. To avoid these, an adhesive to be used in this usage should employ a flexible material capable of relaxing the stress caused by difference in coefficient of thermal expansion between the adhesive and the adherend.

Specifically, polymers to be used in these fields require workability and flexibility for easy bonding with devices and boards, for high degree of freedom in layout, for stress relaxation, and for good handleability. They also require satisfactory thermal stability in terms of heatresistant temperature of higher than 260° C.

Patent Literature (PTL) 1 and PTL 2 disclose 3-ethyl-3-(meth)acryloyloxymethyloxetane and other compounds having an oxetane ring and a (meth)acryloyl group per molecule. These compounds, however, give cured products through polymerization, which cured products disadvantageously have poor flexibility, although having satisfactory thermal stability. PTL 3 also discloses compounds having an oxetane ring and a (meth)acryloyl group per molecule. However, these compounds give cured products through polymerization, which are not always satisfactory in thermal stability and flexibility.

PTL 4 and PTL 5 disclose glycidyl (meth)acrylate, 3,4-epoxycyclohexylmethyl (meth)acrylate, and other epoxy compounds having an epoxy group and a (meth)acryloyl group per molecule. The epoxy compounds, however, have poor curability, exhibit skin irritation and/or toxicity, and are disadvantageous in workability. These compounds give, through polymerization, cured products which fail to have sufficiently satisfactory flexibility. Thus, no resin has been found under present circumstances, which resin is capable of forming a cured product which has satisfactory workability and excels in flexibility and thermal stability.

### CITATION LIST

### Patent Literature

PTL 1: Japanese Unexamined Patent Application Publication (JP-A) No. H11-315181
PTL 2: JP-A No. 2001-40205
PTL 3: JP-A No. 2001-81182
PTL 4: JP-A No. 2005-97515
PTL 5: JP-A No. 2009-242242

### SUMMARY OF INVENTION

### Technical Problem

Accordingly, an object of the present invention is to provide a radically polymerizable resin and a radically polymerizable resin composition which have low viscosities, exhibit excellent workability, can be rapidly cured upon application of heat and/or light to give cured products having satisfactory flexibility and thermal stability. Another object of the present invention is to provide cured products obtained therefrom.

### Solution to Problem

After intensive investigations to achieve the objects, the present inventors have found that a specific resin has a low viscosity and exhibits excellent workability. This resin is obtained by cationic polymerization of a specific monomer alone or in combination with another monomer having a functional group reactive with oxetane ring, which specific monomer includes two different functional groups per molecule, i.e., a radically polymerizable (meth)acryloyl group and a cationically polymerizable oxetane ring linked to each other through an alkylene group with a specific structure. The present inventors have also found that this resin, when further radically polymerized, gives a cured product which excels both in thermal stability and flexibility. The present invention has been made based on these findings.

Specifically, the present invention provides, in an aspect, a radically polymerizable resin obtained through cationic polymerization of an oxetane-ring-containing (meth)acrylic ester compound alone or in combination with another cationically polymerizable compound, in which the oxetane-ring-containing (meth)acrylic ester compound is represented by following Formula (1):

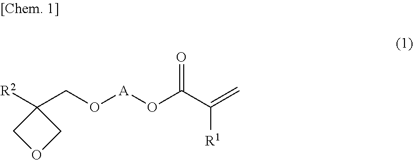

wherein R¹ represents hydrogen atom or methyl group; R² represents hydrogen atom or an alkyl group; and “A” represents a linear or branched chain alkylene group having 4 to 20 carbon atoms.

The other cationically polymerizable compound is preferably a compound having only one functional group selected from the group consisting of oxetane ring, epoxy ring, vinyl ether group, and a vinylaryl group per molecule.

The present invention provides, in another aspect, a radically polymerizable resin composition including the radically polymerizable resin as a radically polymerizable compound.

The radically polymerizable resin composition preferably further include another radically polymerizable compound than the radically polymerizable resin, and the other radically polymerizable compound than the radically polymerizable resin is preferably a compound having one or more functional groups selected from the group consisting of (meth)acryloyl groups, (meth)acryloyloxy groups, (meth)acryloylamino groups, vinylaryl groups, vinyl ether groups, and vinyloxycarbonyl groups per molecule.

The present invention provides, in yet another aspect, a cured product obtained through radical polymerization of the radically polymerizable resin composition.

The cured product is preferably in the form of a film or fiber.

### Advantageous Effects of Invention

The radically polymerizable resin according to the present invention is a resin obtained by cationic polymerization of an oxetane-ring-containing (meth)acrylic ester compound with a specific structure alone or in combination with another cationically polymerizable compound. The resin is therefore has low viscosity and exhibits excellent workability. A radically polymerizable resin composition including the radically polymerizable resin according to the present invention rapidly forms a cured product upon application of heat and/or light. The resulting cured product has satisfactory flexibility and can be bent freely upon usage to exhibit a stress relaxation activity. The cured product also has satisfactory thermal stability to be usable in solder reflow mounting (particularly in lead-free solder mounting) and less suffers from thermal degradation caused by solder reflow. The radically polymerizable resin according to the present invention is therefore usable in the fields of waveguides (e.g., optical waveguides and hybrid substrates), optical fibers, stress-relaxation adhesives, sealants, underfill materials, ink-jet inks, color filters, nanoimprinting materials, flexible substrates and is particularly advantageously usable in the fields of flexible optical waveguides, flexible adhesives, and underfill materials.

### BRIEF DESCRIPTION OF DRAWINGS

FIG. 1 is an explanatory drawing (schematic view indicating data of thermogravimetry) illustrating how to evaluate the thermal stability of a cured product. Description of Embodiments

### OXETANE-RING-CONTAINING (METH)ACRYLIC ESTER COMPOUND

An oxetane-ring-containing (meth)acrylic ester compound for use in the present invention is represented by Formula (1), in which R¹ represents hydrogen atom or methyl group; R² represents hydrogen atom or an alkyl group; and “A” represents a linear or branched chain alkylene group having 4 to 20 carbon atoms.

The alkyl group as R² in Formula (1) is preferably an alkyl group having 1 to 6 carbon atoms, which is typified by linear alkyl groups such as methyl, ethyl, propyl, butyl, pentyl, and hexyl groups; and branched chain alkyl groups such as isopropyl, isobutyl, s-butyl, t-butyl, isopentyl, s-pentyl, t-pentyl, isohexyl, s-hexyl, and t-hexyl groups, of which alkyl groups having 1 to 3 carbon atoms are more preferred. Above all, the alkyl group as R² herein is particularly preferably methyl group or ethyl group.

Group “A” in Formula (1) represents a linear or branched chain alkylene group having 4 to 20 carbon atoms. The group “A” herein is particularly preferably a linear alkylene group represented by following Formula (a1) or a branched chain alkylene group represented by following Formula (a2). These groups are preferred for the formation of a cured product having both satisfactory thermal stability and excellent flexibility. The rightmost end of Formula (a2) is bonded to the oxygen atom constituting the ester bond.

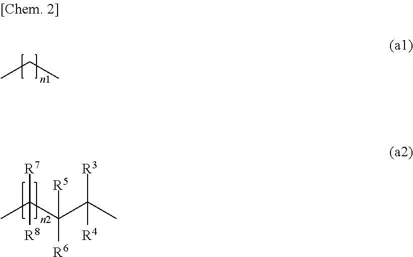

In Formula (a1), n1 denotes an integer of 4 or more. In Formula (a2), R³, R⁴, R⁷, and R⁸ are the same as or different from each other and each represent hydrogen atom or an alkyl group; R⁵ and R⁶ are the same as or different from each other and each represent an alkyl group; and n2 denotes an integer of 0 or more. When n2 is an integer of 2 or more, two or more R⁷s may be the same as or different from one another, and two or more R⁸s may be the same as or different from one another.

Repetition number n1 in Formula (a1) denotes an integer of 4 or more and is preferably an integer of from 4 to 20 and particularly preferably an integer of from 4 to 10. A compound, in which n1 is 3 or less, may give an insufficiently flexible cured product through polymerization.

The alkyl groups as R³, R⁴, R⁵, R⁶, R⁷, and R⁸ in Formula (a2) are preferably alkyl groups having 1 to 4 carbon atoms, which are typified by linear alkyl groups such as methyl, ethyl, propyl, and butyl groups; and branched chain alkyl groups such as isopropyl, isobutyl, s-butyl, and t-butyl groups, of which alkyl groups having 1 to 3 carbon atoms are more preferred. The substituents R³ and R⁴ herein are preferably hydrogen atoms; and R⁵ and R⁶ are each preferably methyl group or ethyl group.

Repetition number n2 in Formula (a2) denotes an integer of 0 or more and is preferably an integer of from 1 to 20 and particularly preferably an integer of from 1 to 10.

Typical examples of the oxetane-ring-containing (meth)acrylic ester compound represented by Formula (1) include the following compounds:

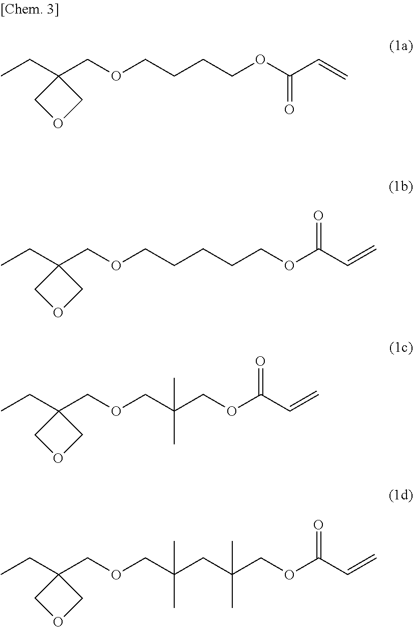

The oxetane-ring-containing (meth)acrylic ester compound represented by Formula (1) may be synthetically prepared typically by allowing a compound represented by following Formula (2):

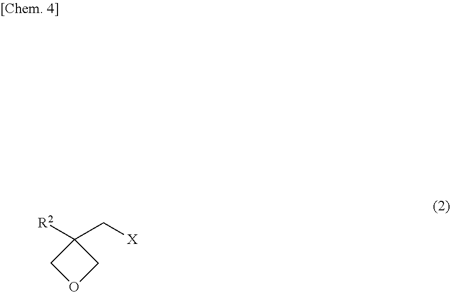

wherein R² is as defined above; and X represents a leaving group,
to react with a compound represented by following Formula (3):

HO-A-OH (3)

wherein “A” is as defined above,
in the presence of a basic substance in a single-liquid phase system to give an oxetane-ring-containing alcohol represented by following Formula (4):

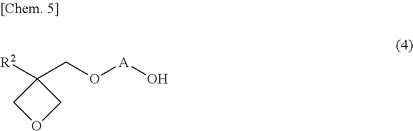

wherein R² and “A” are as defined above; and
(meth)acrylating the oxetane-ring-containing alcohol.

Group X in Formula (2) represents a leaving group, which is typified by groups having high leaving ability, such as chlorine, bromine, and iodine, and other halogen atoms, of which bromine atom and iodine atom are preferred; sulfonyloxy groups such as p-toluenesulfonyloxy group, methanesulfonyloxy group, and trifluoromethanesulfonyloxy group; and carbonyloxy groups such as acetyloxy group.

The basic substance is exemplified by hydroxides of alkali metals or alkaline earth metals, such as sodium hydroxide, potassium hydroxide, calcium hydroxide, and magnesium hydroxide; hydrides of alkali metals or alkaline earth metals, such as sodium hydride, magnesium hydride, and calcium hydride; carbonates of alkali metals or alkaline earth metals, such as sodium carbonate and potassium carbonate; hydrogen carbonates of alkali metals or alkaline earth metals, such as sodium hydrogen carbonate and potassium hydrogen carbonate; and organic metallic compounds including organic lithium reagents (e.g., methyllithium, ethyllithium, n-butyllithium, sec-butyllithium, and ter-butyllithium) and organic magnesium reagents (Grignard reagents; such as CH₃MgBr and C₂H₅MgBr). Each of different basic substances may be used alone or in combination.

As used herein the term “single-liquid phase system” refers to a system including a liquid phase in a number of not two or more but only one. The system may further include a solid, as long as including only one liquid phase. A solvent for use herein may be any one capable of dissolving both the compound represented by Formula (2) and the compound represented by Formula (3). The solvent is typified by aromatic hydrocarbons such as benzene, toluene, xylenes, and ethylbenzene; ethers such as THF (tetrahydrofuran) and IPE (isopropyl ether); sulfur-containing solvents such as DMSO (dimethyl sulfoxide); and nitrogen-containing solvents such as DMF (dimethylformamide).

[Radically Polymerizable Resin]

The radically polymerizable resin according to the present invention is obtained through cationic polymerization of the oxetane-ring-containing (meth)acrylic ester compound represented by Formula (1) alone or in combination with another cationically polymerizable compound. The other cationically polymerizable compound is a compound which is cationically polymerizable and is other than the oxetane-ring-containing (meth)acrylic ester compound represented by Formula (1). This compound is hereinafter also referred to as “other cationically polymerizable compound.”

The oxetane-ring-containing (meth)acrylic ester compound represented by Formula (1) has, per molecule, an oxetane ring serving as a cationically polymerizable moiety and a (meth)acryloyl group serving as a radically polymerizable moiety and can synthetically give a radically polymerizable resin represented by the following formula, through cationic polymerization alone or cationic copolymerization in combination with another cationically polymerizable compound. As used herein the term cationic copolymerization includes block copolymerization and random copolymerization:

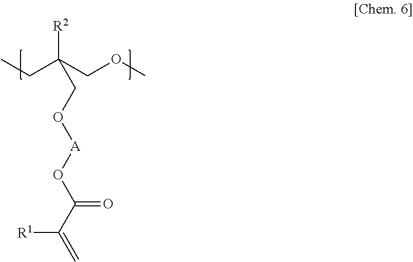

wherein R¹, R², and “A” is as defined above.

The radically polymerizable resin according to the present invention is preferably a resin obtained by cationic copolymerization of the oxetane-ring-containing (meth)acrylic ester compound represented by Formula (1) and the other cationically polymerizable compound, in which a monomer derived from the oxetane-ring-containing (meth)acrylic ester compound represented by Formula (1) account for preferably 0.1 percent by weight or more, more preferably 1 to 99 percent by weight, and particularly preferably 10 to 80 percent by weight, of total monomers constituting the radically polymerizable resin. This is preferred for the formation of a more flexible cured product.

Exemplary other cationically polymerizable compounds include compounds having one or more cationically polymerizable groups such as oxetane rings, epoxy rings, vinyl ether groups, and vinylaryl groups per molecule.

Exemplary compounds having one or more oxetane rings per molecule include trimethylene oxide, 3,3-bis(vinyloxymethyl)oxetane, 3-ethyl-3-hydroxymethyloxetane, 3-ethyl-3-(2-ethylhexyloxymethyl)oxetane, 3-ethyl-3-[(phenoxy)methyl]oxetane, 3-ethyl-3-(hexyloxymethyl)oxetane, 3-ethyl-3-(chloromethyl)oxetane, 3,3-bis(chloromethyl)oxetane, 1,4-bis[(3-ethyl-3-oxetanylmethoxy)methyl]benzene, bis{[1-ethyl(3-oxetanyl)]methyl}ether, 4,4′-bis[(3-ethyl-3-oxetanyl)methoxymethyl]bicyclohexyl, 1,4-bis[(3-ethyl-3-oxetanyl)methoxymethyl]cyclohexane, and 3-ethyl-3-[[(3-ethyloxetan-3-yl)methoxy]methyl]oxetane; and derivatives of them.

Exemplary compounds having one or more epoxy rings per molecule include ethers such as glycidyl methyl ether, bisphenol-A diglycidyl ether, bisphenol-F diglycidyl ether, bisphenol-S diglycidyl ether, brominated bisphenol-A diglycidyl ethers, brominated bisphenol-F diglycidyl ethers, brominated bisphenol-S diglycidyl ethers, epoxy novolak resins, hydrogenated bisphenol-A diglycidyl ethers, hydrogenated bisphenol-F diglycidyl ethers, hydrogenated bisphenol-S diglycidyl ethers, 3,4-epoxycyclohexylmethyl 3′,4′-epoxycyclohexanecarboxylate, 2-(3,4-epoxycyclohexyl-5,5-spiro-3,4-epoxy)cyclohexane-meta-dioxane, bis(3,4-epoxycyclohexylmethyl)adipate, bis(3,4-epoxy-6-methylcyclohexylmethyl)adipate, 3,4-epoxy-6-methylcyclohexyl 3′,4′-epoxy-6′-methylcyclohexanecarboxylate, methylenebis(3,4-epoxycyclohexane), dicyclopentadiene diepoxide, di(3,4-epoxycyclohexylmethyl)ether of ethylene glycol, ethylenebis(3,4-epoxycyclohexanecarboxylate), dioctyl epoxyhexahydrophthalate, di-2-ethylhexyl epoxyhexahydrophthalate, 1,4-butanediol diglycidyl ether, 1,6-hexanediol diglycidyl ether, glycerol triglycidyl ether, trimethylolpropane triglycidyl ether, polyethylene glycol diglycidyl ethers, and polypropylene glycol diglycidyl ethers; polyglycidyl ethers of polyetherpolyols obtained by addition of one or more different alkylene oxides to an aliphatic polyhydric alcohol such as ethylene glycol, propylene glycol, or glycerol; diglycidyl esters of aliphatic long-chain dibasic acids; monoglycidyl ethers of aliphatic higher alcohols; monoglycidyl ethers of phenol, cresol, butylphenol, or a polyether alcohol obtained by addition of an alkylene oxide thereto; and fatty acid glycidyl esters, such as (R)-glycidyl butyrate, as well as derivatives of them.

Exemplary compounds having one or more vinyl ether groups per molecule include 2-hydroxyethyl vinyl ether, 3-hydroxypropyl vinyl ether, 2-hydroxypropyl vinyl ether, 2-hydroxyisopropyl vinyl ether, 4-hydroxybutyl vinyl ether, 3-hydroxybutyl vinyl ether, 2-hydroxybutyl vinyl ether, 3-hydroxyisobutyl vinyl ether, 2-hydroxyisobutyl vinyl ether, 1-methyl-3-hydroxypropyl vinyl ether, 1-methyl-2-hydroxypropyl vinyl ether, 1-hydroxymethylpropyl vinyl ether, 4-hydroxycyclohexyl vinyl ether, 1,6-hexanediol monovinyl ether, 1,4-cyclohexanedimethanol monovinyl ether, 1,3-cyclohexanedimethanol monovinyl ether, 1,2-cyclohexanedimethanol monovinyl ether, p-xylene glycol monovinyl ether, m-xylene glycol monovinyl ether, o-xylene glycol monovinyl ether, diethylene glycol monovinyl ether, triethylene glycol monovinyl ether, tetraethylene glycol monovinyl ether, pentaethylene glycol monovinyl ether, oligoethylene glycol monovinyl ethers, polyethylene glycol monovinyl ethers, dipropylene glycol monovinyl ether, tripropylene glycol monovinyl ether, tetrapropylene glycol monovinyl ether, pentapropylene glycol monovinyl ether, oligopropylene glycol monovinyl ethers, and polypropylene glycol monovinyl ethers, as well as derivative of them.

Exemplary compounds having one or more vinylaryl groups per molecule include styrene, divinylbenzene, methoxystyrene, ethoxystyrene, hydroxystyrene, vinylnaphthalene, vinylanthracene, 4-vinylphenyl acetate, (4-vinylphenyl)dihydroxyborane, (4-vinylphenyl)boranic acid, (4-vinylphenyl)boronic acid, 4-ethenylphenylboronic acid, 4-vinylphenylboranic acid, 4-vinylphenylboronic acid, p-vinylphenylboric acid, p-vinylphenylboronic acid, N-(4-vinylphenyl)maleimide, N-(p-vinylphenyl)maleimide, and N-(p-vinylphenyl)maleimide, as well as derivatives of them.

Of other cationically polymerizable compounds for use herein, preferred are compounds having only one functional group selected from the group consisting of oxetane ring, epoxy ring, vinyl ether group, and a vinylaryl group per molecule. These compounds are preferred for the formation of a satisfactorily flexible and thermally stable cured product having excellent flexibility and satisfactory thermal stability. Among them, more preferred are compounds having only one oxetane ring per molecule, such as trimethylene oxide, 3-ethyl-3-(2-ethylhexyloxymethyl)oxetane, 3-ethyl-3-[(phenoxy)methyl]oxetane, and 3-ethyl-3-(hexyloxymethyl)oxetane; and compounds having only one epoxy group per molecule, such as glycidyl methyl ether and (R)-glycidyl butyrate. Each of them may be used alone or in combination.

The cationic polymerization reaction may be performed in the presence of a solvent. The solvent is exemplified by benzene, toluene, and xylenes.

The cationic polymerization reaction may employ a polymerization initiator. The polymerization initiator is not limited, as long as capable of inducing cationic polymerization, and can be any of known or customary cationic polymerization initiators and acid generators. These are typified by protonic acids such as perchloric acid, sulfuric acid, phosphoric acid, p-toluenesulfonic acid, trichloroacetic acid, and trifluoroacetic acid; Lewis acids such as boron trifluoride, aluminum bromide, aluminum chloride, antimony pentachloride, ferric chloride, tin tetrachloride, titanium tetrachloride, mercury chloride, and zinc chloride; as well as iodine and triphenylchloromethane. Each of them may be used alone or in combination.

The polymerization initiators may be used in the cationic polymerization reaction in an amount of typically from about 0.01 to about 50 percent by weight, and preferably from about 0.1 to about 20 percent by weight, relative to the total amount of cationically polymerizable compounds (total weight of the oxetane-ring-containing (meth)acrylic ester compound represented by Formula (1) and the other cationically polymerizable compounds).

The cationic polymerization reaction may be performed in the presence of a polymerization inhibitor. The polymerization inhibitor is typified by qunone/penol inhibitors such as 4-methoxyphenol, hydroquinone, methylhydroquinone, dimethylhydroquinone, trimethylhydroquinone, hydroquinone monomethyl ether, 2,5-di-tert-butylhydroquinone, p-tert-butylcatechol, mono-t-butylhydroquinone, p-benzoquinone, naphthoquinone, 2,5-di-tert-butyl-p-cresol, α-naphthol, and nitrophenol; thioether inhibitors; and phosphite inhibitors.

The radically polymerizable resin has a weight-average molecular weight of typically 500 or more (e.g., from about 500 to about 1000000), and preferably from 3000 to 500000. The radically polymerizable resin, if having a weight-average molecular weight less than the above-specified range, may give an insufficiently flexible cured product through radical polymerization.

[Radically Polymerizable Resin Composition]

A radically polymerizable resin composition according to an embodiment of the present invention includes the radically polymerizable resin as a radically polymerizable compound.

The radically polymerizable resin composition includes the radically polymerizable resin in a content of typically 5 percent by weight or more and may substantially include the radically polymerizable resin alone. The radically polymerizable resin composition contains the radically polymerizable resin in a content of preferably 10 percent by weight or more, and particularly preferably from 60 to 90 percent by weight, for the formation of a more flexible cured product. The radically polymerizable resin composition, if containing the radically polymerizable resin in a content of less than 5 percent by weight, may give an insufficiently flexible cured product by curing through radical polymerization.

The radically polymerizable resin composition according to the present invention may further contain, in addition to the radically polymerizable resin, a compound which is radically polymerizable but is other than the oxetane-ring-containing (meth)acrylic ester compound represented by Formula (1). This compound is hereinafter also referred to as “other radically polymerizable compound”).

The other radically polymerizable compound is typified by compounds having one or more radically polymerizable groups such as (meth)acryloyl groups, (meth)acryloyloxy groups, (meth)acryloylamino groups, vinylaryl groups, vinyl ether groups, and vinyloxycarbonyl groups per molecule.

Exemplary compounds having one or more (meth)acryloyl groups per molecule include 1-buten-3-one, 1-penten-3-one, 1-hexen-3-one, 4-phenyl-1-buten-3-one, and 5-phenyl-1-penten-3-one; as well as derivatives of them.

Exemplary compounds having one or more (meth)acryloyloxy groups per molecule include methyl (meth)acrylate, ethyl (meth)acrylate, n-butyl (meth)acrylate, isobutyl (meth)acrylate, t-butyl methacrylate, 2-ethylhexyl (meth)acrylate, isodecyl (meth)acrylate, n-lauryl (meth)acrylate, n-stearyl (meth)acrylate, n-butoxyethyl (meth)acrylate, butoxydiethylene glycol (meth)acrylate, methoxytriethylene glycol (meth)acrylate, methoxypolyethylene glycol (meth)acrylate, cyclohexyl (meth)acrylate, n-hexyl (meth)acrylate, tetrahydrofurfuryl (meth)acrylate, benzyl (meth)acrylate, phenoxyethyl (meth)acrylate, isobornyl (meth)acrylate, 2-hydroxyethyl (meth)acrylate, 2-hydroxypropyl (meth)acrylate, 2-hydroxybutyl (meth)acrylate, dimethylaminoethyl (meth)acrylate, diethylaminoethyl (meth)acrylate, methacrylic acid, 2-methacryloyloxyethyl succinate, 2-methacryloyloxyethyl hexahydrophthalate, methacryloyloxyethyl-2-hydroxypropyl phthalate, glycidyl (meth)acrylate, 2-methacryloyloxyethyl acid phosphate (2-hydroxyethyl methacrylate phosphate), ethylene glycol di(meth)acrylate, diethylene glycol di(meth)acrylate, triethylene glycol di(meth)acrylate, 1,4-butanediol di(meth)acrylate, neopentyl glycol di(meth)acrylate, 1,6-hexanediol di(meth)acrylate, 1,9-nonanediol di(meth)acrylate, 1,10-decanediol di(meth)acrylate, glycerol di(meth)acrylate, 2-hydroxy-3-acryloyloxypropyl (meth)acrylate, dimethyloltricyclodecane di(meth)acrylate, trifluoroethyl (meth)acrylate, perfluorooctylethyl (meth)acrylate, isoamyl (meth)acrylate, isomyristyl (meth)acrylate, γ-(meth)acryloxypropyltrimethoxysilane, 2-(meth)acryloyloxyethyl isocyanate, 1,1-bis(acryloyloxy)ethyl isocyanate, 2-(2-methacryloyloxyethyloxy)ethyl isocyanate, vinyitrimethoxysilane, vinyltriethoxysilane, and 3-(meth)acryloxypropyltriethoxysilane; as well as derivatives of them.

Exemplary compounds having one or more (meth)acryloylamino groups per molecule include morpholin-4-yl acrylate, acryloylmorpholine, N,N-dimethylacrylamide, N,N-diethylacrylamide, N-methylacrylamide, N-ethylacrylamide, N-propylacrylamide, N-isopropylacrylamide, N-butylacrylamide, N-n-butoxymethylacrylamide, N-hexylacrylamide, and N-octylacrylamide; as well as derivatives of them.

Exemplary compounds having one or more vinylaryl groups per molecule and exemplary compounds having one or more vinyl ether groups per molecule are as with those mentioned as the other cationically polymerizable compounds.

Exemplary compounds having one or more vinyloxycarbonyl groups per molecule include isopropenyl formate, isopropenyl acetate, isopropenyl propionate, isopropenyl butyrate, isopropenyl isobutyrate, isopropenyl caproate, isopropenyl valerate, isopropenyl isovalerate, isopropenyl lactate, vinyl acetate, vinyl propionate, vinyl butyrate, vinyl caproate, vinyl caprylate, vinyl laurate, vinyl myristate, vinyl palmitate, vinyl stearate, vinyl cyclohexanecarboxylate, vinyl pivalate, vinyl octanoate, vinyl monochloroacetate, divinyl adipate, vinyl methacrylate, vinyl crotonate, vinyl sorbate, vinyl benzoate, and vinyl cinnamate; as well as derivatives of them.

Of other radically polymerizable compounds for use herein, preferred are compounds having two or more (particularly preferably two) (meth)acryloyloxy groups, such as ethylene glycol di(meth)acrylate, diethylene glycol di(meth)acrylate, triethylene glycol di(meth)acrylate, 1,4-butanediol di(meth)acrylate, neopentyl glycol di(meth)acrylate, 1,6-hexanediol di(meth)acrylate, 1,9-nonanediol di(meth)acrylate, 1,10-decanediol di(meth)acrylate, and glycerol di(meth)acrylate. These compounds are preferred for the formation of a further thermally stable cured product. Each of them may be used alone or in combination.

The radically polymerizable resin composition according to the present invention preferably contains the radically polymerizable resin in combination with the other radically polymerizable compound, for the formation of a further thermally stable cured product. The compositional ratio of the radically polymerizable resin to the other radically polymerizable compound in terms of weight ratio [(the former):(the latter)] is typically from 95:5 to 5:95, preferably from 95:5 to 20:80, and particularly preferably from 95:5 to 60:40. The radically polymerizable resin composition, if containing the radically polymerizable resin in a compositional ratio out of the above-specified range, may give an insufficiently flexible cured product.

The radically polymerizable resin composition according to the present invention may contain, but not exclusively, a polymerization initiator. The polymerization initiator can be any of known or customary thermal polymerization initiators, photo-induced radical-polymerization initiators, and other polymerization initiators capable of inducing radical polymerization.

The thermal polymerization initiators are exemplified by benzoyl peroxide, azobisisobutyronitrile (AIBN), azobis-2,4-dimethylvaleronitrile, and dimethyl 2,2′-azobis(isobutyrate). Each of them may be used alone or in combination.

The photo-induced radical-polymerization initiators are typified by benzophenone, acetophenone benzil, benzil dimethyl ketone, benzoin, benzoin methyl ether, benzoin ethyl ether, benzoin isopropyl ether, dimethoxyacetophenone, dimethoxyphenylacetophenone, diethoxyacetophenone, and diphenyl disulfite. Each of them may be used alone or in combination.

The polymerization initiator may be used in combination with a synergistic agent to enhance the conversion of photo-adsorbed energy to polymerization-initiating free radicals. The synergistic agent is typified by amines such as triethylamine, diethylamine, diethanolamine, ethanolamine, dimethylaminobenzoic acid, and methyl dimethylaminobenzoate; and ketones such as thioxanthone, 2-isopropylthioxanthone, 2,4-diethylthioxanthone, and acetylacetone.

The radically polymerizable resin composition contains, if any, a polymerization initiator in an amount of from about 0.01 to about 50 percent by weight, and preferably from about 0.1 to about 20 percent by weight, relative to the total amount of radically polymerizable compounds (total weight of the radically polymerizable resin and the other radically polymerizable compound) contained therein.

The radically polymerizable resin composition according to the present invention may further contain one or more other additives according to necessity, within ranges not adversely affecting advantageous effects of the present invention. Exemplary other additives include known or customary additives such as setting-expandable monomers, photosensitizers (e.g., anthracene sensitizers), resins, adhesion promoters, reinforcers, softeners, plasticizers, viscosity modifiers, solvents, inorganic or organic particles (e.g., nano-scale particles), and fluorosilanes.

The radically polymerizable resin composition according to the present invention, when subjected to a heating treatment and/or light irradiation to promote the radical polymerization reaction, can form a cured product. The heating treatment, when employed, may be performed at a temperature of typically from about 20° C. to about 200° C., preferably from about 50° C. to about 150° C., and more preferably from about 70° C. to about 120° C. The temperature, however, may be suitably controlled according to the types of components to be reacted and of a catalyst. The light irradiation, when employed, may use any of light sources such as mercury lamps, xenon lamps, carbon arc lamps, metal halide lamps, sunlight, electron beams, and laser beams. The light irradiation may be followed by a heating treatment at a temperature of typically from about 50° C. to 180° C. to allow curing to proceed.

The radical polymerization reaction may be performed under normal atmospheric pressure, under reduced pressure, or under a pressure (under a load). The reaction may be performed in any atmosphere, such as air atmosphere, nitrogen atmosphere, or argon atmosphere, as long as not adversely affecting the reaction.

The cured product obtained through radical polymerization of the radically polymerizable resin composition according to the present invention is not limited in its shape or form and may be in the form of a film or fiber. A cured product in the form of a film (film-like cured product) may be produced typically by applying the radically polymerizable resin composition to a substrate (base material) using an applicator so as to have a uniform thickness, and applying heat and/or light to promote the radical polymerization reaction. A cured product in the form of a fiber (fibrous cured product) may be produced typically by quantitatively extruding the radically polymerizable resin composition using a syringe, and applying heat and/or light to the extruded radically polymerizable resin composition to promote the radical polymerization reaction.

The resulting cured product is satisfactorily flexible and highly thermally stable. The radically polymerizable resin composition according to the present invention is therefore useful in the fields of waveguides (e.g., optical waveguides and hybrid substrates), optical fibers, stress-relaxation adhesives, sealants, underfill materials, ink-jet inks, color filters, nanoimprinting materials, and flexible substrates and is particularly useful in the fields of flexible optical waveguides, flexible adhesives, and underfill materials.

### EXAMPLES

The present invention will be illustrated in further detail with reference to several working examples below. It should be noted, however, that these examples are never construed to limit the scope of the present invention.

### Example 1

### Production of Radically Polymerizable Resin

A mixture (monomer mixture) of 3.78 g of toluene, 8.82 g (34.67 mmol) of 3-ethyl-3-(3-acryloyloxy-2,2-dimethylpropyloxymethyl)oxetane (EOXTM-NPAL) represented by following formula, and 0.039 g of 4-methoxyphenol was placed in a three-necked flask equipped with an initiator-dropping line, a nitrogen line, and a thermometer, followed by temperature adjustment to 25° C. Next, a mixture of 0.093 g of toluene and 0.016 g (0.11 mmol) of boron trifluoride diethyl ether complex was quantitatively added dropwise over 2 hours using a delivery pump. After the completion of dropwise addition, this was held for 4 hours, purified by precipitation from five times the amount of methanol (containing 0.1% of 4-methoxyphenol), held in a vacuum dryer (40° C., full vacuum) for 20 hours, and yielded a colorless, transparent liquid resin (1).

The liquid resin (1) had a weight-average molecular weight of 24800 in terms of a polystyrene standard.

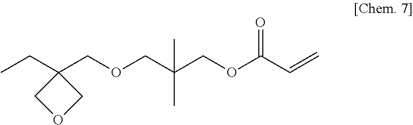 [SMILES: C=CC(=O)OCC(C)(C)COCC1(CC)COC1]

### Example 2

### Production of Radically Polymerizable Resin

A mixture (monomer mixture) of 19.98 g of toluene, 8.82 g (34.67 mmol) of EOXTM-NPAL, 23.56 g (0.10 mol) of 3-ethyl-3-(2-ethylhexyloxymethyl)oxetane (trade name “OXT-212,” supplied by Toagosei Co., Ltd.), and 0.039 g of 4-methoxyphenol was placed in a three-necked flask equipped with an initiator-dropping line, a nitrogen line, and a thermometer, followed by temperature adjustment to 25° C. Next, a mixture of 5.60 g of toluene and 0.95 g (6.60 mmol) of boron trifluoride diethyl ether complex was quantitatively added dropwise over 2 hours using a delivery pump. After the completion of dropwise addition, this was held for 4 hours, purified by precipitation from five times the amount of methanol (containing 0.1% of 4-methoxyphenol), held in a vacuum dryer (40° C., full vacuum) for 20 hours, and yielded a colorless, transparent liquid resin (2).

The liquid resin (2) had a weight-average molecular weight of 8200 in terms of a polystyrene standard.

### Example 3

### Production of Radically Polymerizable Resin

A mixture (monomer mixture) of 20.40 g of toluene, 8.82 g (34.67 mmol) of EOXTM-NPAL, 39.26 g (0.17 mol) of OXT-212, and 0.039 g of 4-methoxyphenol was placed in a three-necked flask equipped with an initiator-dropping line, a nitrogen line, and a thermometer, followed by temperature adjustment to 25° C. Next, a mixture of 5.60 g of toluene and 0.95 g (6.60 mmol) of boron trifluoride diethyl ether complex was quantitatively added dropwise over 2 hours using a delivery pump. After the completion of dropwise addition, this was held for 4 hours, purified by precipitation from five times the amount of methanol (containing 0.1% of 4-methoxyphenol), held in a vacuum dryer (40° C., full vacuum) for 20 hours, and yielded a colorless, transparent liquid resin (3).

The liquid resin (3) had a weight-average molecular weight of 8300 in terms of a polystyrene standard.

### Example 4

### Production of Radically Polymerizable Resin

A mixture (monomer mixture) of 20.40 g of toluene, 8.33 g (34.44 mmol) of 3-ethyl-3-(4-acryloyloxybutyloxymethyl)oxetane (EOXTM-BAL) represented by the following formula, 39.26 g (0.17 mol) of OXT-212, and 0.039 g of 4-methoxyphenol was placed in a three-necked flask equipped with an initiator-dropping line, a nitrogen line, and a thermometer, followed by temperature adjustment to 25° C. Next, a mixture of 5.60 g of toluene and 0.95 g (6.60 mmol) of boron trifluoride diethyl ether complex was quantitatively added dropwise over 2 hours using a delivery pump. After the completion of dropwise addition, this was held for 4 hours, purified by precipitation from five times the amount of methanol (containing 0.1% of 4-methoxyphenol), held in a vacuum dryer (40° C., full vacuum) for 20 hours, and yielded a colorless, transparent liquid resin (4).

The liquid resin (4) had a weight-average molecular weight of 34800 in terms of a polystyrene standard.

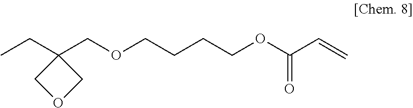 [SMILES: C=CC(=O)OCCCCOCC1(CC)COC1]

### Comparative Example 1

### Production of Radically Polymerizable Resin

A mixture (monomer mixture) of 3.78 g of toluene, 5.85 g (34.77 mmol) of 3-ethyl-3-acryloyloxymethyloxetane (trade name “OXT-10,” supplied by Osaka Organic Chemical Industry Ltd.), and 0.039 g of 4-methoxyphenol was placed in a three-necked flask equipped with an initiator-dropping line, a nitrogen line, and a thermometer, followed by temperature adjustment to 25° C. Next, a mixture of 0.093 g of toluene and 0.016 g (0.11 mmol) of boron trifluoride diethyl ether complex was quantitatively added dropwise over 2 hours using a delivery pump. After the completion of dropwise addition, this was held for 4 hours, purified by precipitation from five times the amount of methanol (containing 0.1% of 4-methoxyphenol), held in a vacuum dryer (40° C., full vacuum) for 20 hours, and yielded a colorless, transparent liquid resin (5).

The liquid resin (5) had a weight-average molecular weight of 11900 in terms of a polystyrene standard.

### Examples 5 to 50 and Comparative Examples 2 to 4

Radically polymerizable resin compositions were prepared by compounding components in blending ratios as given in Tables 1 to 3 below. Numerical values in these tables are indicated by part by weight.

Evaluations

The radically polymerizable resin compositions obtained in Examples 5 to 50 and Comparative Examples 2 to 4 were polymerized in the following manner to give cured products, and flexibility and thermal stability of the resulting cured products were evaluated.

[Formation (1) of Film-Like Cured Products]

Each of the radically polymerizable resin compositions obtained in Examples 5 to 27 and Comparative Examples 2 to 4 was applied to a non-silicone release film (trade name “T789,” supplied by Daicel Value Coating Ltd.) as a substrate, another ply of the non-silicone release film (trade name “T789,” supplied by Daicel Value Coating Ltd.) as a substrate was pressed thereon while interposing spacers between the two release films so as to allow the distance between the two release films to be about 100 μm, and a heating treatment at 85° C. for one hour was applied thereto to yield film-like cured products each about 100 μm thick.

[Formation (2) of Film-Like Cured Products]

Each of the radically polymerizable resin compositions obtained in Examples 28 to 50 was applied to a non-silicone release film (“T789,” supplied by Daicel Value Coating Ltd.) as a substrate, so as to have a thickness of about 100 μm, and another ply of the non-silicone release film (trade name “T789,” supplied by Daicel Value Coating Ltd.) as a substrate was pressed thereon while interposing spacers between the two release films so as to allow the distance between the two release films to be about 100 μm. The article was irradiated with ultraviolet rays at an irradiation energy of about 2 J and a wavelength of 320 to 390 nm using a belt-conveyer type ultraviolet irradiator (UVC-02516S1AA02, supplied by Ushio Inc.), and thereby yielded film-like cured products each about 100 μm thick.

[Formulation of Fibrous Cured Products]

Each of the radically polymerizable resin compositions obtained in Examples 28 to 50 was charged into a syringe, quantitatively extruded therefrom (at a rate of 3 mL/second), the extruded radically polymerizable resin compositions was irradiated with ultraviolet rays at an irradiation energy of 1.5 W/cm² per one direction and a wavelength of 365 nm, and thereby yielded fibrous cured products each 50 to 2000 μm in diameter.

[Evaluation of Flexibility]

Each of the film-like cured products about 100 μm thick produced through curing of the radically polymerizable resin compositions obtained in Examples 5 to 50 and Comparative Examples 2 to 4 was placed around a rod, whether the samples suffered from cracks (cracking) or not was visually observed, and the flexibility of the samples was evaluated according to the following criteria.

Criteria:

Very good (VG): Sample did not suffer from cracks (cracking) even when placed around a rod 1 mm in radius;

Good: Sample did not suffer from cracks (cracking) when placed around a rod 2 mm in radius; and

Poor: Sample suffered from cracks (cracking) when placed around a rod 2 mm in radius

[Evaluation of Thermal Stability]

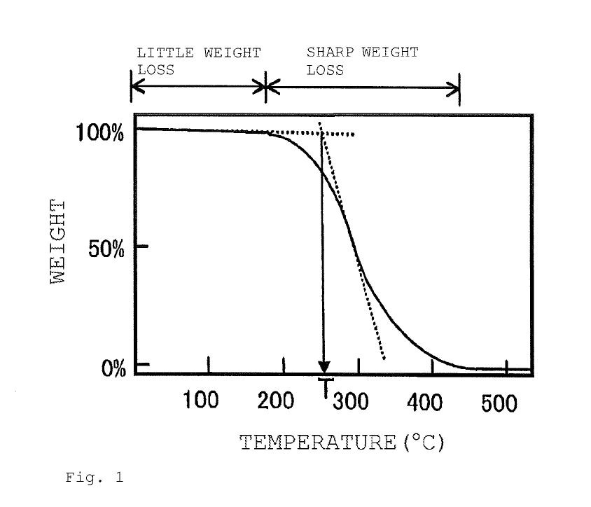

Each of the film-like cured products about 100 μm thick produced through curing of the radically polymerizable resin compositions obtained in Examples 5 to 50 and Comparative Examples 2 to 4 was subjected to thermogravimetry using a thermal analyzer (trade name “TG-DTA6300,” supplied by Seiko Instruments Inc.). With reference to FIG. 1, a temperature at the point, where the tangent line of a region with no weight loss or gradual weight loss in early stages of temperature rise intersects with the tangent line of the inflection point of a region with abrupt weight loss, was defined as a pyrolysis temperature T (° C.), and the thermal stability of the sample was evaluated according to the following criteria.

Criteria:

Good: Sample had a pyrolysis temperature T (° C.) of 260° C. or higher; and

Poor: Sample had a pyrolysis temperature T of lower than 260° C.

TABLE 1

| | | Examples | | | | | | | | | | | | | | | | |
|---|---|---|---|---|---|---|---|---|---|---|---|---|---|---|---|---|---|---|
| | | 5 | 6 | 7 | 8 | 9 | 10 | 11 | 12 | 13 | 14 | 15 | 16 | 17 | 18 | 19 | 20 | 21 |
| Radically | Liquid | 100 | | | | 90 | | | | 80 | | | | 70 | | | | |
| polymerizable | resin (1) | | | | | | | | | | | | | | | | | |
| resins | Liquid | | 100 | | | | 90 | | | | 80 | | | | 70 | | | |
| | resin (2) | | | | | | | | | | | | | | | | | |
| | Liquid | | | 100 | | | | 90 | | | | 80 | | | | 70 | | |
| | resin (3) | | | | | | | | | | | | | | | | | |
| | Liquid | | | | 100 | | | | 90 | | | | 80 | | | | 70 | 60 |
| | resin (4) | | | | | | | | | | | | | | | | | |
| | Liquid | | | | | | | | | | | | | | | | | |
| | resin (5) | | | | | | | | | | | | | | | | | |
| Other radically | B1 | | | | | 10 | 10 | 10 | 10 | 20 | 20 | 20 | 20 | 30 | 30 | 30 | 30 | 20 |
| polymerizable | B2 | | | | | | | | | | | | | | | | | 20 |
| compounds | | | | | | | | | | | | | | | | | | |
| Inorganic microparticles | | | | | | | | | | | | | | | | | | |
| General- | PS | | | | | | | | | | | | | | | | | |
| purpose | PMMA | | | | | | | | | | | | | | | | | |
| resins | | | | | | | | | | | | | | | | | | |
| Polymerization | V601 | 3 | 3 | 3 | 3 | 3 | 3 | 3 | 3 | 3 | 3 | 3 | 3 | 3 | 3 | 3 | 3 | 3 |
| initiators | Benzo- | | | | | | | | | | | | | | | | | |
| | phenone | | | | | | | | | | | | | | | | | |
| Evaluations | Thermal | Good | Good | Good | Good | Good | Good | Good | Good | Good | Good | Good | Good | Good | Good | Good | Good | Good |
| | stability | | | | | | | | | | | | | | | | | |
| | Flexibility | Good | Good | Good | Good | VG | VG | VG | VG | VG | VG | VG | VG | VG | VG | VG | VG | Good |

TABLE 2

| | | Examples | | | | | | | | | | | | | | | | |
|---|---|---|---|---|---|---|---|---|---|---|---|---|---|---|---|---|---|---|
| | | 22 | 23 | 24 | 25 | 26 | 27 | 28 | 29 | 30 | 31 | 32 | 33 | 34 | 35 | 36 | 37 | 38 |
| Radically | Liquid | | | | | | | 100 | | | | 90 | | | | 80 | | |
| polymerizable | resin (1) | | | | | | | | | | | | | | | | | |
| resins | Liquid | | | | | | | | 100 | | | | 90 | | | | 80 | |
| | resin (2) | | | | | | | | | | | | | | | | | |
| | Liquid | | | | | | | | | 100 | | | | 90 | | | | 80 |
| | resin (3) | | | | | | | | | | | | | | | | | |
| | Liquid | 40 | 20 | 38 | 36 | 28 | 20 | | | | 100 | | | | 90 | | | |
| | resin (4) | | | | | | | | | | | | | | | | | |
| | Liquid | | | | | | | | | | | | | | | | | |
| | resin (5) | | | | | | | | | | | | | | | | | |
| Other radically | B1 | 30 | 40 | 29 | 27 | 21 | 15 | | | | | 10 | 10 | 10 | 10 | 20 | 20 | 20 |
| polymerizable | B2 | 30 | 40 | 29 | 27 | 21 | 15 | | | | | | | | | | | |
| compounds | | | | | | | | | | | | | | | | | | |
| Inorganic microparticles | | | | 4 | 10 | 30 | 50 | | | | | | | | | | | |
| General- | PS | | | | | | | | | | | | | | | | | |
| purpose | PMMA | | | | | | | | | | | | | | | | | |
| resins | | | | | | | | | | | | | | | | | | |
| Polymerization | V601 | 3 | 3 | 3 | 3 | 3 | 3 | | | | | | | | | | | |
| initiators | Benzo- | | | | | | | 3 | 3 | 3 | 3 | 3 | 3 | 3 | 3 | 3 | 3 | 3 |
| | phenone | | | | | | | | | | | | | | | | | |
| Evaluations | Thermal | Good | Good | Good | Good | Good | Good | Good | Good | Good | Good | Good | Good | Good | Good | Good | Good | Good |
| | stability | | | | | | | | | | | | | | | | | |
| | Flexibility | Good | Good | Good | Good | Good | Good | Good | Good | Good | Good | VG | VG | VG | VG | VG | VG | VG |

TABLE 3

| | | | | | | | | | | | | | | Comparative | | |
|---|---|---|---|---|---|---|---|---|---|---|---|---|---|---|---|---|
| | | Examples | | | | | | | | | | | | Examples | | |
| | | 39 | 40 | 41 | 42 | 43 | 44 | 45 | 46 | 47 | 48 | 49 | 50 | 2 | 3 | 4 |
| Radically | Liquid resin (1) | | 70 | | | | | | | | | | | | | |
| polymerizable resins | Liquid resin (2) | | | 70 | | | | | | | | | | | | |
| | Liquid resin (3) | | | | 70 | | | | | | | | | | | |
| | Liquid resin (4) | 80 | | | | 70 | 60 | 40 | 20 | 38 | 36 | 28 | 20 | | | |
| | Liquid resin (5) | | | | | | | | | | | | | | | |
| Other radically | B1 | 20 | 30 | 30 | 30 | 30 | 20 | 30 | 40 | 29 | 27 | 21 | 15 | | | |
| polymerizable | B2 | | | | | | 20 | 30 | 40 | 29 | 27 | 21 | 15 | | | |
| compounds | | | | | | | | | | | | | | | | |
| Inorganic microparticles | | | | | | | | | | 4 | 10 | 30 | 50 | | | |
| General-purpose resins | PS | | | | | | | | | | | | | | 100 | |
| | PMMA | | | | | | | | | | | | | | | 100 |
| Polymerization | V601 | | | | | | | | | | | | | 3 | | |
| initiators | Benzophenone | 3 | 3 | 3 | 3 | 3 | 3 | 3 | 3 | 3 | 3 | 3 | 3 | | | |
| Evaluations | Thermal stability | Good | Good | Good | Good | Good | Good | Good | Good | Good | Good | Good | Good | Good | Poor | Poor |
| | Flexibility | VG | VG | VG | VG | VG | Good | Good | Good | Good | Good | Good | Good | Poor | Poor | Poor |

Symbols and abbreviations in the tables represent the following compounds:

B1: 1,10-Bis(acryloyloxy)decane [i.e., 1,10-decanediol diacrylate] (supplied by Wako Pure Chemical Industries, Ltd.)

B2: Dodecyl acrylate [i.e., lauryl acrylate] (supplied by Wako Pure Chemical Industries, Ltd.)

Inorganic microparticles: Surface-treated silica (trade name “SC2500-SVJ,” supplied by Admatechs Company Limited)

PS: Polystyrene

PMMA: Poly(methyl methacrylate)

V601: Dimethyl 2,2′-azobis(isobutyrate) (trade name “V601,” supplied by Wako Pure Chemical Industries, Ltd.)

1. A radically polymerizable resin obtained through cationic polymerization of an oxetane-ring-containing (meth)acrylic ester compound alone or in combination with another cationically polymerizable compound, the oxetane-ring-containing (meth)acrylic ester compound being represented by following Formula (1): 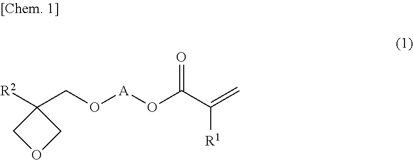
wherein R¹ represents hydrogen atom or methyl group; R² represents hydrogen atom or an alkyl group; and “A” represents a linear or branched chain alkylene group having 4 to 20 carbon atoms.

2. The radically polymerizable resin according to claim 1, wherein the other cationically polymerizable compound is a compound having only one functional group selected from the group consisting of oxetane ring, epoxy ring, vinyl ether group, and a vinylaryl group per molecule.

3. A radically polymerizable resin composition comprising the radically polymerizable resin of claim 1 as a radically polymerizable compound.

4. The radically polymerizable resin composition according to claim 3, further comprising another radically polymerizable compound than the radically polymerizable resin.

5. The radically polymerizable resin composition according to claim 4, wherein the other radically polymerizable compound than the radically polymerizable resin is a compound having one or more functional groups selected from the group consisting of (meth)acryloyl groups, (meth)acryloyloxy groups, (meth)acryloylamino groups, vinylaryl groups, vinyl ether groups, and vinyloxycarbonyl groups per molecule.

6. A cured product obtained through radical polymerization of the radically polymerizable resin composition of claim 3.

7. The cured product according to claim 6, in the form of a film.

8. The cured product according to claim 6, in the form of a fiber.
